(19) United States
(12) Patent Application Publication
(10) Pub. No.: US 2016/0135712 A1
(43) Pub. Date: May 19, 2016

(54) SYSTEM AND METHOD FOR DETERMING THE POSITION OF A CATHETER

(71) Applicant: AngioDynamics, Inc., Latham, NY (US)

(72) Inventors: Kevin Holochwost, Hudson, MA (US); Paul DiCarlo, Middleboro, MA (US)

(73) Assignee: AngioDynamics, Inc., Latham, NY (US)

(21) Appl. No.: 14/937,194

(22) Filed: Nov. 10, 2015

Related U.S. Application Data
(60) Provisional application No. 62/079,094, filed on Nov. 13, 2014.

(51) Int. Cl. A61B 5/06; A61B 5/00

(52) U.S. Cl. CPC A61B 5/062; A61B 5/742; A61B 5/7475

(57) ABSTRACT

A system for locating a catheter includes a catheter with a locatable tip and a hardware platform. The locatable tip can include a magnetic field generator. The hardware can include a plurality of sensor clusters and control circuitry. Each of the sensor clusters can measure a magnetic field generated by the magnetic field generator. The control circuitry can receive signals from the first, second, and third sensors clusters based on the measured magnetic fields and display a graphical representation for a position of the locatable tip.

### CROSS-REFERENCE TO RELATED APPLICATIONS

This application claims priority to U.S. Provisional Application No. 62/079,094, filed on Nov. 13, 2014 and titled Tip Location Direction Sensing through Hall Effects, incorporated herein by reference.

### INCORPORATION BY REFERENCE

The subject matter of co-pending U.S. patent application Ser. No. 14/211,801 filed Mar. 14, 2014 and titled System and Methods for Catheter Tip Placement Using ECG is incorporated herein by reference.

### FIELD OF THE DISCLOSURE

Embodiments of the present disclosure relate generally to systems and methods to determine the position of a catheter, and more particularly to systems and methods for sensing a magnetic field originating from a catheter tip to locate the position of the catheter tip within a patient.

### BACKGROUND OF THE DISCLOSURE

Catheters are tubular medical device that may be inserted in the body of a patient to treat diseases or perform surgical procedures. Medical professionals commonly use catheters for gaining prolonged access to an area within the body of a patient. Once the catheter tip is positioned at the target location, treatments such as antibiotics, chemotherapy, pain medicine, and nutrition can be administered. If the catheter tip is improperly positioned during insertion, various risks to the patient arise, including a fluid infusion that causes pain or injury to the patient, complication due to increased thrombosis rates, delays in therapy, catheter malfunction, and additional costs.

General standards for proper catheter insertion depend of the type of catheter and the treatment being provided. For example, peripherally inserted central catheters (or PICC lines) are commonly inserted into a brachial, cephalic, or basilica vein in the arm and advanced through the venous system towards the superior vena cava (SVC). Current medical standards recommend that the distal tip of the catheter be positioned in the lower third of the SVC, close to the junction of the SVC and the right atrium (RA). However, since PICC lines are commonly inserted into a vein in the arm and advanced through the venous system to reach the SVC, the distal tip of the PICC line may be inadvertently positioned in a non-target area, such as the internal jugular, the subclavian vein, or too far past the SCV-RA junction and into the heart. Therefore, a need in the art exists for an improved device and system that can assist a user, typically a nurse, doctor, or other trained medical technician, when placing a PICC line in a patient's body. Improved PICC line tip placement systems and methods for overcoming these issues are desired and described in more detail below.

### SUMMARY

This summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

An exemplary embodiment of a catheter location device in accordance with the present disclosure can include one or more sensor clusters and control circuitry. Each of the one or more sensor clusters may measure a first set of voltage values when a locatable tip of a catheter is at a first position and a second set of voltage values when the locatable tip in at a second position. The control circuitry may include a processor communicatively coupled to the one or more sensor clusters and a non-transitory computer-readable medium. The processor may execute instructions stored on the non-transitory computer-readable medium to calculate the second position based in part on the first position and in part on a change between the first set of voltage values and the second set of voltage values. The processor may also execute instructions to store the second position of the locatable tip to the non-transitory computer-readable medium.

A exemplary embodiment of a catheter location system is disclosed, and may include a catheter and a hardware platform. The catheter may include a locatable tip with a magnetic field generator. The hardware platform may include one or more sensor clusters and control circuitry. Each of the one or more sensor clusters may measure a magnetic field generated by the magnetic field generator. The control circuitry may receive signals from the one or more sensor clusters based on the magnetic field measured by the one or more sensor clusters and display a graphical representation for a position of the locatable tip.

An exemplary method of locating a catheter tip in accordance with the present disclosure may include: placing a catheter into a patient, the catheter having a locatable tip and a magnetic field generator; measuring movement of the locatable tip using a hardware system, the hardware system comprising control circuitry and one or more sensor clusters, each sensor cluster to measure a magnetic field generated by the magnetic field generator; receiving signals, at the control circuitry, from the one or more sensor clusters based on the magnetic field measured by the one or more sensor clusters; and displaying a graphical representation for a position of the locatable tip.

### BRIEF DESCRIPTION OF THE DRAWINGS

By way of example, various embodiments of the disclosed device will now be described, with reference to the accompanying drawings, in which:

FIG. 1 is an overview of an exemplary catheter location system in conjunction with a patient in accordance with an embodiment of the present disclosure;

FIG. 2A is a schematic of an exemplary catheter with a permanent magnet in accordance with an embodiment of the present disclosure;

FIG. 2B is a schematic of an exemplary catheter with an electromagnet coupled to an exemplary hardware platform in accordance with an embodiment of the present disclosure;

FIG. 3 is a block diagram of an exemplary embodiment of a hardware platform;

FIG. 4 is a block diagram of an exemplary sensor cluster in conjunction with exemplary control circuitry in accordance with an embodiment of the present disclosure;

FIG. 5 is a schematic illustrating an exemplary arrangement of directional sensors in a sensor cluster in accordance with an embodiment of the present disclosure;

FIG. 6A is a schematic of an exemplary locatable tip moving across in accordance with an embodiment of the present disclosure;

FIG. 6B is a schematic of an exemplary locatable tip moving up in accordance with an embodiment of the present disclosure;

FIG. 6C is a schematic of an exemplary locatable tip moving down in accordance with an embodiment of the present disclosure;

FIG. 7A is a flow chart illustrating an exemplary process for determining a position of a locatable tip in accordance with an embodiment of the present disclosure;

FIG. 7B is a flow chart illustrating an exemplary process for determining a position of a locatable tip in accordance with an embodiment of the present disclosure;

FIG. 8 is a logic diagram illustrating an exemplary method of determining a position of a locatable tip in accordance with an embodiment of the present disclosure.

### DETAILED DESCRIPTION

The present embodiments will now be described more fully hereinafter with reference to the accompanying drawings, in which some embodiments are shown. The subject matter of the present disclosure, however, may be embodied in many different forms and should not be construed as limited to the embodiments set forth herein. Rather, these embodiments are provided so that this disclosure will be thorough and complete, and will fully convey the scope of the subject matter to those skilled in the art. In the drawings, like numbers refer to like elements throughout.

In the following description, numerous specific details are set forth. However, it is understood that embodiments of the invention may be practiced without these specific details. In other instances, well-known methods, structures, and techniques have not been shown in detail in order not to obscure an understanding of this description.

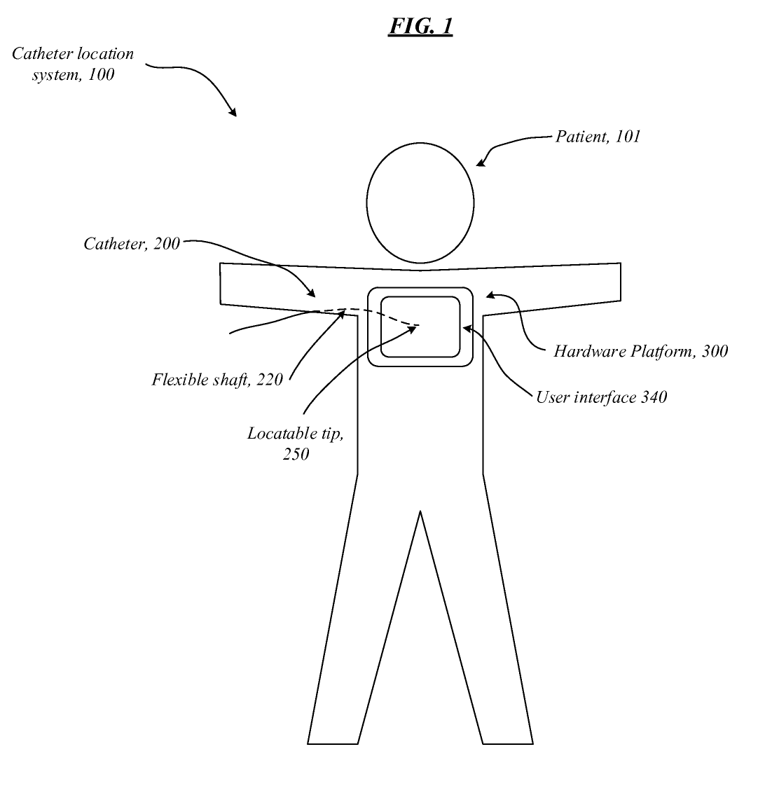

Referring to FIG. 1, an exemplary embodiment of a catheter location system 100 in conjunction with a patient 101 is shown. In general, the catheter location system 100 includes a catheter 200 and hardware platform 300. The catheter 200 may include a flexible shaft 220 and a locatable tip 250. The hardware platform 300 may include a user interface 340. The catheter 200 may include any commonly known catheter in the art including, but not limited to, a PICC line, a midline catheter, a central venous catheter (CVC), dialysis catheter, drainage catheter, or any other catheter that may require visual assistance when being placed in the patient by the user.

The catheter 200 may be placed (inserted) in the patient 101 by a user for administering medications, withdrawing fluids, performing a medical procedure, or as a preventative measure. To place the catheter 200, the locatable catheter tip 250 may be inserted into a blood vessel of the patient 101 using standard access techniques currently known in the art. The flexible shaft 220 provides tractability to the catheter 200 and allows the locatable tip 250 to move along the lumen of the blood vessel, enabling the user to advance the locatable tip 250 towards a treatment position within the patient 101. In one embodiment the catheter 200 is a peripherally inserted central catheter (PICC) being placed over a guidewire, and the treatment position is the superior vena cava (SVC) junction of the heart.

The hardware platform 300 may approximate one or more positions of the locatable tip 250, as the catheter 200 is being placed in the patient 101. The one or more approximated positions of the locatable tip 250 may be communicated to the user via the user interface 340. In some embodiments the user interface 340 may display a graphical representation for a position of the locatable tip 250. Providing the user with the approximated position of the catheter 200 within the patient 101 can assist the user in properly placing the catheter 200 within the patient 101 and directing the catheter 200 towards the treatment site in a safe and efficient manner. The user interface 240 may include a display such as an LED or LCD to communicate the one or more approximated positions. In some embodiments the user interface may include a touchscreen display for receiving input from the user. Alternatively, the user interface 240 may also be a downloadable software application that can be uploaded onto a portable tablet computer, smart phone, or other wireless device.

In the illustrated embodiment of FIG. 11 the hardware platform 300 is placed on or directly above the chest of the patient 101 to approximate positions of the locatable tip 250. However, one having ordinary skill in the art will appreciate that the hardware platform 300 and any of its components can be configured to approximate positions of the locatable tip 250 in any number of orientations relative to the patient 101 without departing from the scope of the present disclosure.

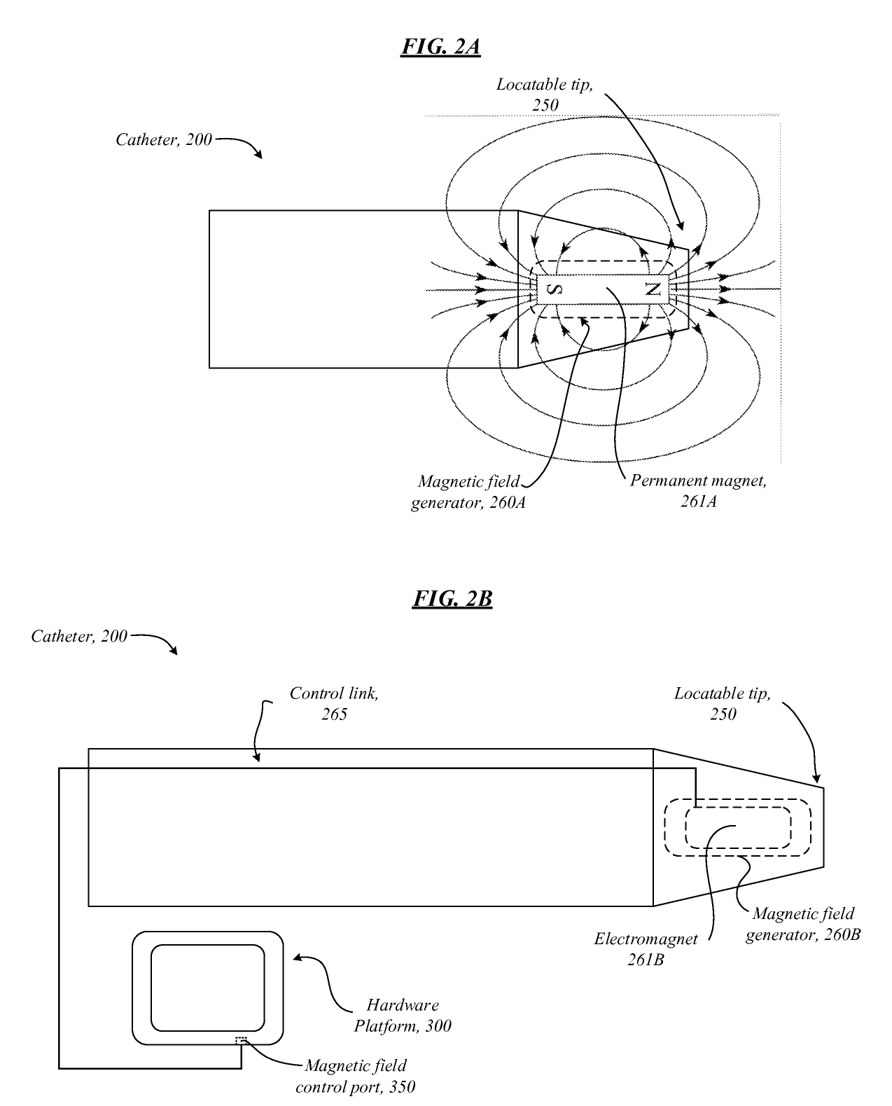

Referring now to FIG. 2A, an exemplary embodiment of the catheter 200 with a permanent magnet 261A is shown. The catheter 200 can include locatable tip 250 and a magnetic field generator 260A with the permanent magnet 261A. The permanent magnet 261A may have north and south poles and generate a substantially constant magnetic field. In other embodiments the magnetic field may vary in magnitude and/or direction. Examples of a variable magnetic field will be discussed in more detail below with respect to FIG. 2B. The permanent magnet 261A may be embedded into the locatable tip 250. In some embodiments, the permanent magnet 261A may form a portion of a stylet attached to the catheter 200 proximate the locatable tip 250.

In the illustrated embodiment the permanent magnet 261A is arranged such that the north and south poles are generally in line along the length of the catheter 200 with the north pole closer to the locatable tip 250. Alternatively, the permanent magnet 261A is arranged such that the south pole is closer to the locatable tip 250. In some embodiments the end of the permanent magnet 261A is at least 2 cm from the locatable tip 250. Although the permanent magnet 261A is arranged in the orientation described, those of ordinary skill in the art will appreciate that the permanent magnet 261A may be arranged in any number of orientations with respect to the locatable tip 250 as long as the orientation is known or determinable.

The substantially constant magnetic field can be visualized with magnetic field lines emerging from the north pole of the permanent magnet 261A and reentering at the south pole. The hardware platform 300 (FIG. 1) may use the substantially constant magnetic field to safely and efficiently approximate one or more positions of the locatable tip 250 as the catheter 200 is moved within a patient 101 (FIG. 1). Once the catheter 200 is placed within the patient 101 (FIG. 1), desired medical interventions may be safely and effectively delivered to the patient 101.

Referring now to FIG. 2B, the catheter 200 with an electromagnet 261B coupled to an exemplary hardware platform 300 in accordance with an exemplary embodiment of the present disclosure is shown. The catheter 200 can include locatable tip 250, and a magnetic field generator 260B with the electromagnet 261B. The electromagnet 261B may be coupled to the hardware platform 300 via a control link 265 and generate a variable magnetic field. The variable magnetic field may change in magnitude and/or direction to create a desired magnetic field. The electromagnet 261B may be embedded into a wall of the catheter 200, such as a wall of the locatable tip 250. In some embodiments, the electromagnet 261B may form a portion of a stylet attached to the catheter 200 proximate the locatable tip 250.

The electromagnet 261B may generate the desired magnetic field based on electrical signals received from the hardware platform 300 via the control link 265. In the illustrated embodiment the control link 265 couples to the hardware platform 300 via a magnetic field control port 350. In other embodiments the control link 265 may include a wireless communication module and/or a power supply. For example, the wireless communication module may receive wireless signals from the hardware platform 300 to control the electromagnet 261B by altering the output of the power supply.

The variable magnetic field may be used by the hardware platform 300 (FIG. 1) to approximate one or more positions of the locatable tip 250 as the catheter 200 is moved within a patient 101 (FIG. 1). In some embodiments varying the magnetic field may increase the safety and/or accuracy of placing the catheter 200. For example, increasing the magnetic field may improve the accuracy of an approximated position of the locatable tip 250. In another example, decreasing the magnetic field may improve the safety while placing the catheter 200 by reducing or eliminating adverse effects of the magnetic field such as on other medical devices such as a defibrillator or pump implanted in the patient 101.

Once the catheter 200 is properly placed at the treatment site, desired medical interventions may be safely and effectively delivered to the patient 101. In some embodiments the catheter 200 may have multiple lumens. For example, medical interventions may be delivered via a first lumen, while the control link 265 may be in a second lumen allowing the control link 265 to couple to the magnetic field control port 350 of the hardware platform 300 without entering or contaminating the first lumen. In another example the catheter may include a third lumen to prevent a guidewire from contaminating a medical intervention. Alternatively, the control link 265 may be embedded in the catheter wall.

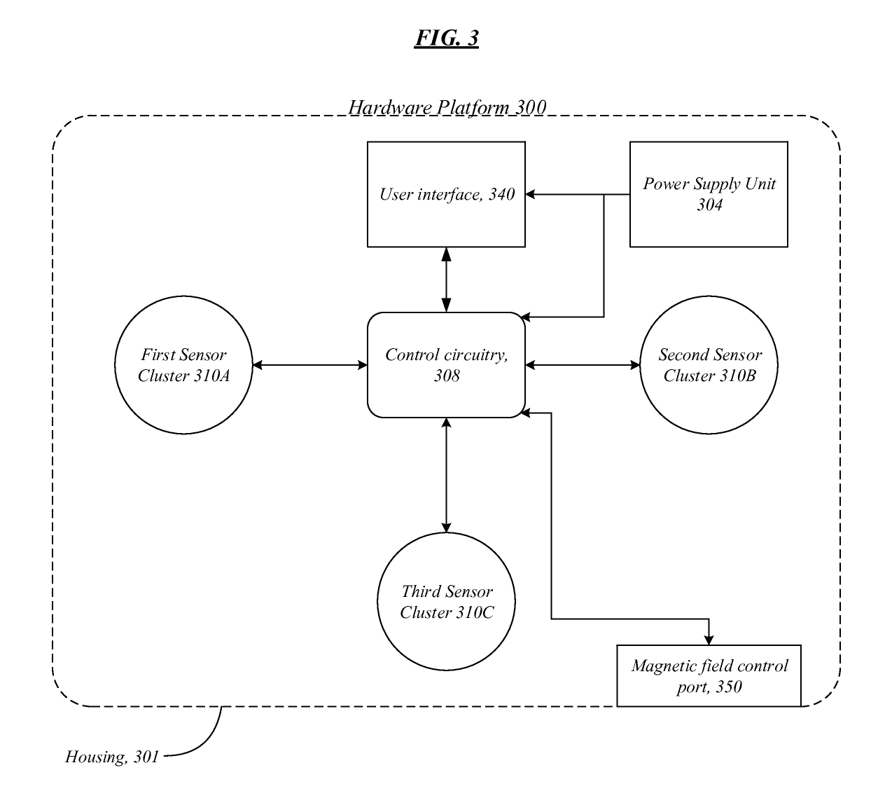

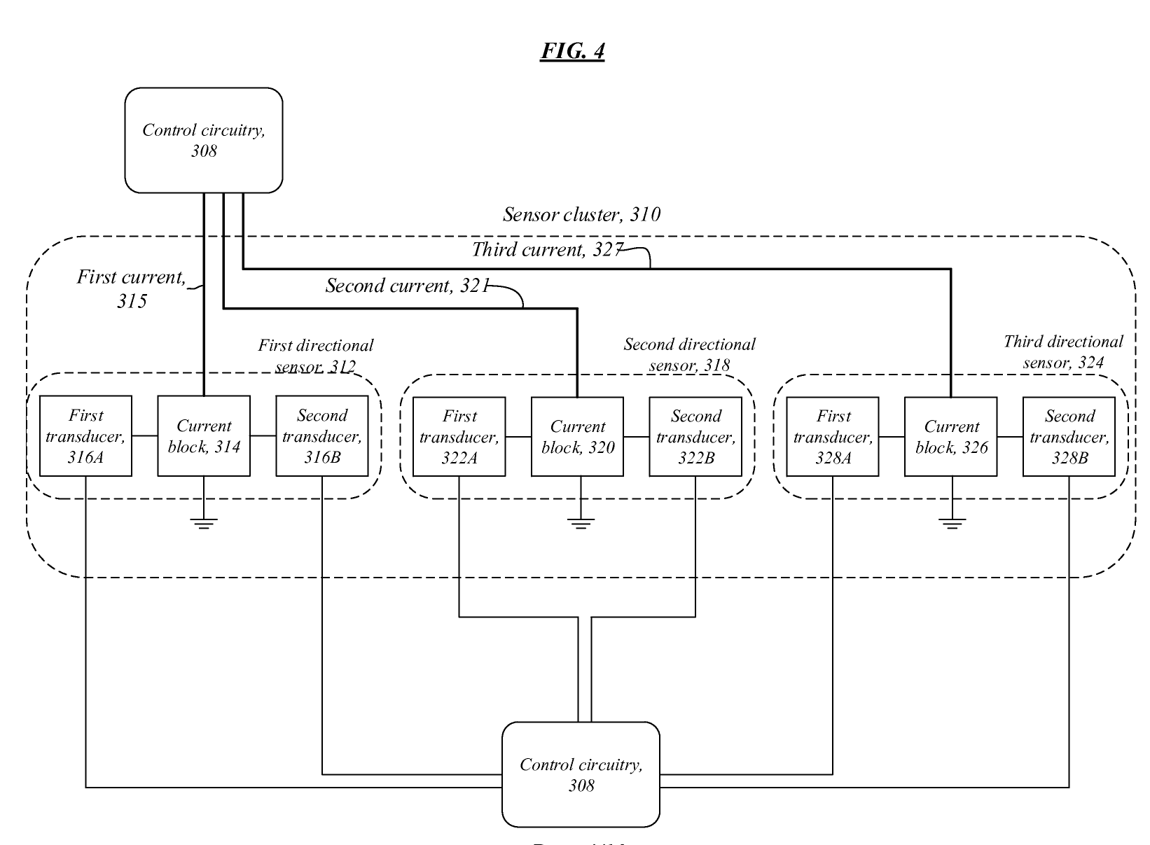

Referring now to FIG. 3, an exemplary embodiment of the hardware platform 300 is shown. The hardware platform 300 may include housing 301, power supply unit 304, control circuitry 308, a first sensor cluster 310A, a second sensor cluster 310B, a third sensor cluster 310C, the user interface 340, and magnetic field control port 350. The control circuitry 308 may interpret data from the sensor clusters 310A-C to approximate a position of the locatable tip 250 of the catheter 200 within a patient 101 (FIG. 1). The housing 301 may enclose and/or protect one or more components of the hardware platform 300. In some embodiments one or more components, such as the sensor clusters, may be separate from the hardware platform 300. As will be appreciated by those of ordinary skill in the art, although the illustrated embodiment includes three sensor clusters, other embodiments may include one or more sensor clusters without departing from the scope of the present disclosure.

The power supply unit 304 may provide an electrical current to operate the components of the hardware platform. The control circuitry 308 may operate to monitor and/or control one or more components of the hardware platform 300 and/or catheter 200 (FIG. 1). The control circuitry 308 may effect control over one or more components of the hardware platform 300 through communicative couplings such as a conductive wire carrying electrical signals between one or more components of the catheter system 100. Alternatively one or more of the components may communicate wirelessly. In some embodiments one or more commands to direct control of the hardware platform 300 may be received by the control circuitry 308 through the user interface 340. For example, a graphical user interface (GUI) may enable a user to direct control of the hardware platform 300.

The first, second, and third sensor clusters 310A-C may output a set of data or values in response to a magnetic field. The magnetic field may originate from the catheter 200 (FIG. 1). In some embodiments the strength or direction of the magnetic field may be controlled by the control circuitry 308 through the magnetic field control port 350. However, as will be appreciated by one of ordinary skill in the art, the hardware platform 300 may have more, less, or different components than those described herein. For example, when the catheter 200 utilizes a permanent magnet 261A as described in conjunction with FIG. 2A, the magnetic field control port 350 may not be necessary. In another example, a different number of sensor clusters, such as 2 or 4, may be used. In a third example, the hardware platform 300 may include TILO or Celerity hardware as described in U.S. patent application Ser. No. 14/211,801 filed Mar. 14, 2014 and titled System and Methods for Catheter Tip Placement Using ECG, which is incorporated herein by reference.

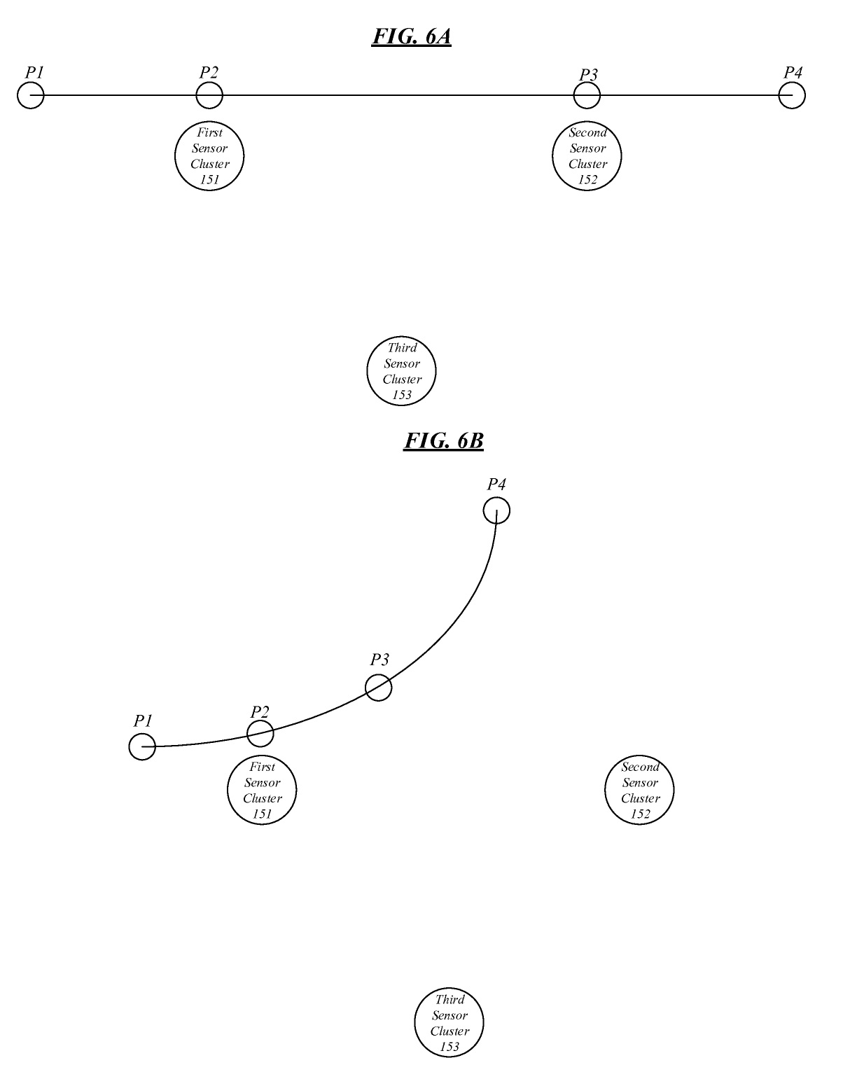

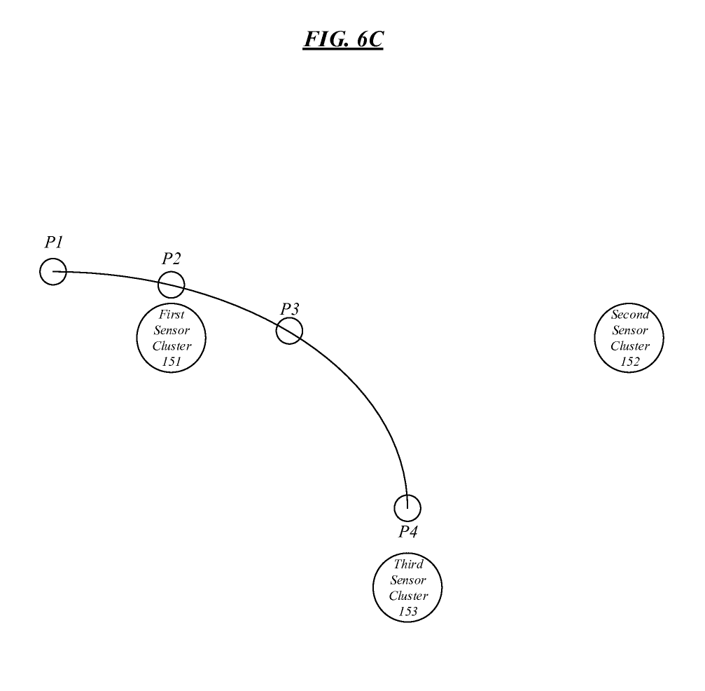

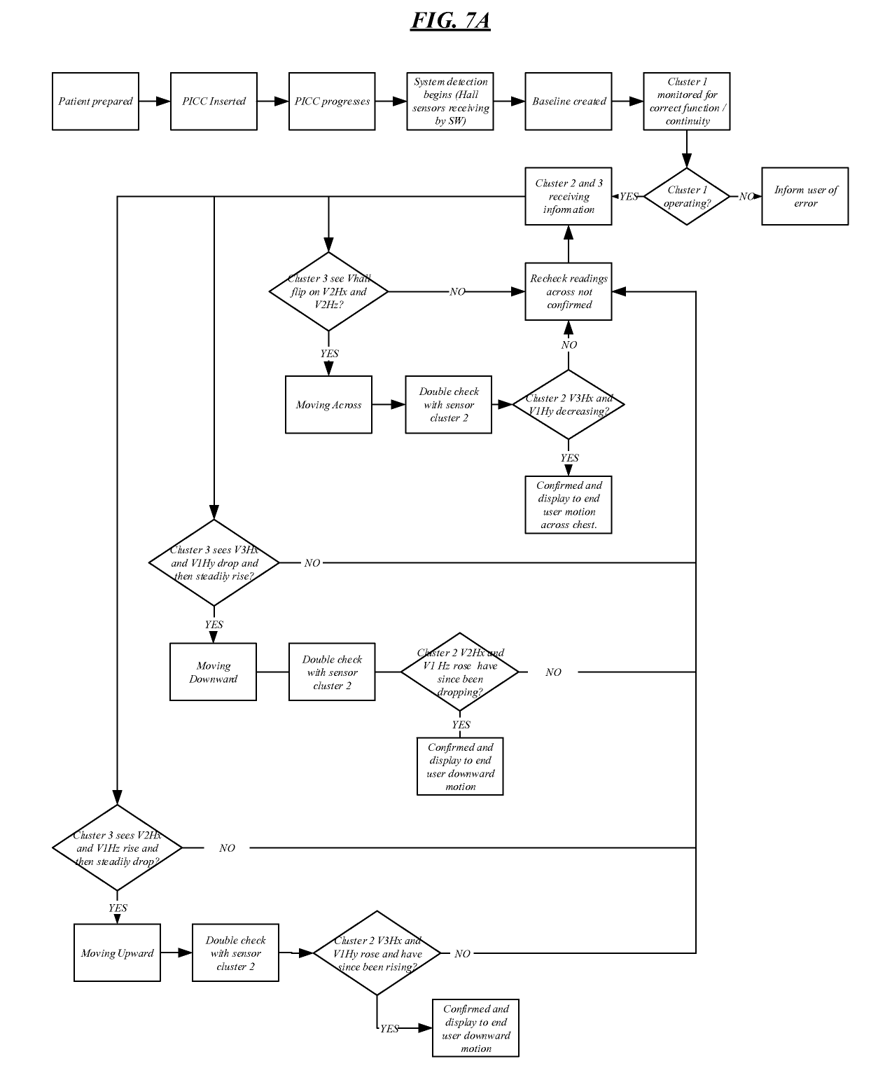

The control circuitry 308 may include logic to approximate a position of the catheter 200 based on the set of data or values output by one or more of the sensor clusters 310A-C. The set of data or values output may correspond to a set of voltages measured by one or more of the sensor clusters 310A-C in response to a magnetic field. In various embodiments the control circuitry 308 may include a non-transitory computer-readable medium to store operational data and/or computer-readable instructions such as the position approximation logic. The position approximation logic will be described in more detail with respect to FIGS. 6A-8. The control circuitry 308 may also include a processor to execute the computer-readable instructions and store or manipulate the operational data. The operational data may include a series of sets of data or values over time. The sets of data or values may indicate a magnitude or direction of the magnetic field. In some embodiments each sensor cluster may include a plurality of transducers, with each transducer measuring an effect of the magnetic field.

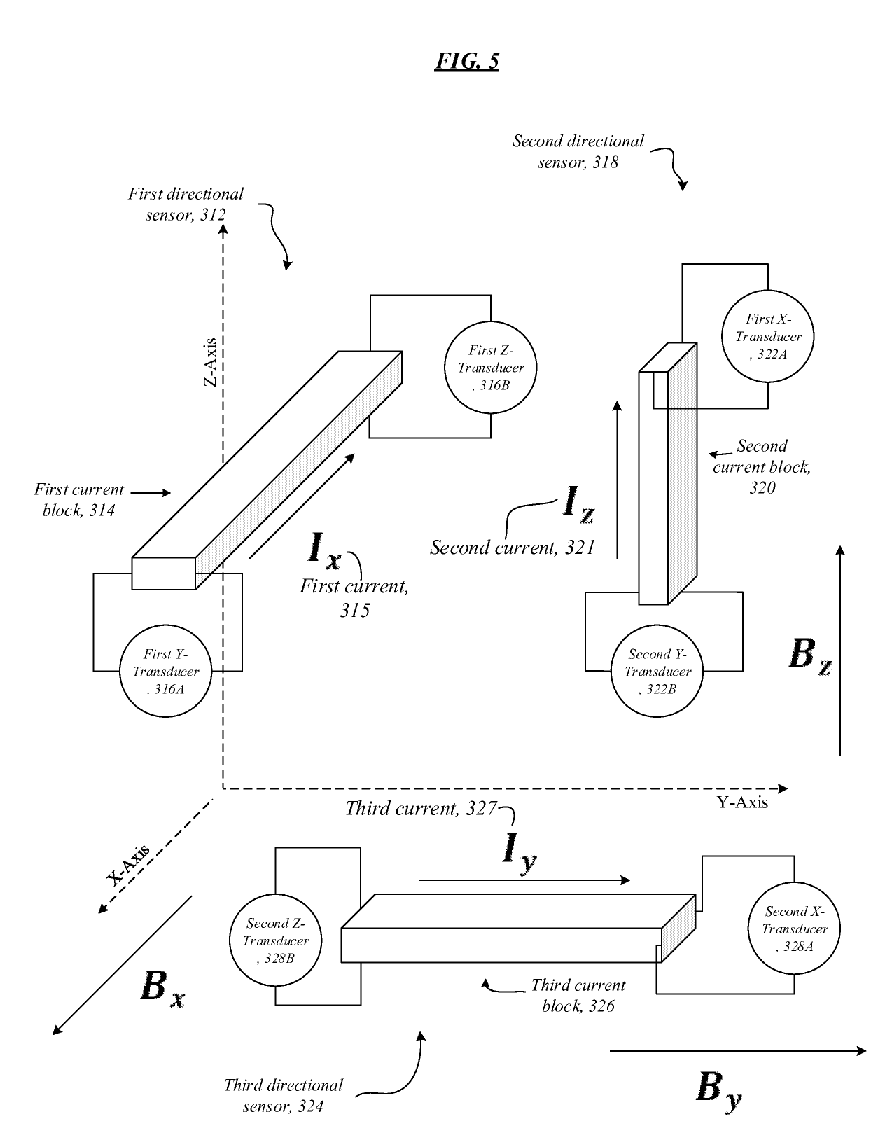

Referring now to FIG. 4, an exemplary embodiment of a sensor cluster 310 is shown. Each sensor cluster 310 may include three directional sensors 312, 318, 324. Each direction sensor 312, 318, 324 may include a current block 314, 320, 326 coupled to first and second transducers 316A-B, 322A-B, 328A-B. In some embodiments the transducers 316A-B, 322A-B, 328A-B are voltage sensors. The control circuitry may provide first, second, and third currents 315, 321, 327 to the current blocks 314, 320, 326. The first transducers 316A, 322A, 328A may sense an effect, in a first dimension, a magnetic field has on the respective currents 315, 321, 327 flowing through the current blocks 314, 320, 326. The second transducers 316B, 322B, 328B may sense an analogous effect, in a second dimension, the magnetic field has on the respective currents 315, 321, 327. The first and second dimensions may be orthogonal with respect to each other.

In one non-limiting exemplary embodiment the first and second transducers 316A-B, 322A-B, 328A-B detect the Hall Effect on the currents 315, 321, 327 as they pass through respective current blocks 314, 320, 326. The transducers may output the Hall Effect as a set of voltages or similar values. The output values may be stored as operational data and later processed by control circuitry 308 (FIG. 3). Although the illustrated embodiment includes three sensor clusters, other embodiments may use one or more sensor clusters.

The Hall Effect is the redistribution of charges in a conductor, such as current blocks 314, 320, 326, due to the presence of a magnetic field. As electrons or positive holes move across the conductor, they represent a current, such as currents 315, 321, 327. This current may be deflected by the magnetic field according to the right hand rule and the usual magnetic force defined by the Lorentz Force Equation (1):

F=q(\vec{E}+\vec{V}×\vec{B}) (1)

As a positive charge moves across the conductor there will be a force due to the magnetic field (2):

F=q(V̅_̅{̅d̅}̅B_{⊥}) (2)

As the charge begins to pile on one side of the wire, there will be a reacting force from the built up charge to oppose the magnetic field, which will be the electric force. When they balance there will be a steady state (3). This occurs rather rapidly. In some embodiments, steady state may be required to approximate a position of the catheter 200 (FIG. 1).

F_{E}=F_{B} (3)

### Rewritten as:

q(\vec{E})=q(\vec{V}×\vec{B}) (4)

Reduced by drift velocity to:

E=V_{d}B_{⊥} (5)

The electric field in a steady state will be defined as the hall voltage across the width of the wire in the direction of the field in question:

$$E = \frac{{V}_{H}}{{w}_{wire}} \qquad ( 6 )$$

Relate the current and drift velocity to one another by the definition of current:

I=ρ_{η}qν_{d}A (7)

Fill all known values into the steady state definition (5):

$$\frac{{V}_{H}}{{w}_{wire}} = \frac{{IB}_{⊥}}{{ρ}_{η} qA} \qquad ( 8 )$$

Assume a spherical conductor:

$${V}_{H} = \frac{{IB}_{⊥}}{{ρ}_{η} q π r} \qquad ( 9 )$$

This defines the hall voltage across the conductor for a perpendicular magnetic field. This allows voltage sensors across the conductor, such as first and second transducers 316A-B, 322A-B, 328A-B, to be used to measure with precision both the applied magnetic field and the current in a circuit. With this three “knob,” system of the current, the voltage, and the magnetic field, if any pair is known the other one can be derived. In some embodiments, the current will be controlled and the relative magnitude of the magnetic field will then be used to map the hall voltages in three dimensions with three directional sensors 312, 318, 324. The mapped hall voltages may be processed by logic circuitry 308 to determine a position of the catheter 200.

Referring now to FIG. 5, an exemplary arrangement of directional sensors 312, 318, 324 in sensor cluster 310 in accordance with an embodiment of the present disclosure is shown. The sensor cluster 310 may include directional sensors 312, 318, 324 with current blocks 314, 620, 326 and transducers 316A-B, 322A-B, 328A-B for each current 315, 321, 327, respectively. The directional sensors and/or transducers may include one or more hall sensors. The position of the catheter 200 (FIG. 1) may be delineated, at least in part, by easily calculable changes in voltages measured by transducers 316A-B, 322A-B, 328A-B based on the geometry illustrated in FIG. 5.

This behavior enables a system in which each hall sensor obeys the rules of Eqn. (9) and the relative magnitudes of the hall sensors obey Table 1.

TABLE 1

| | Sensor Effect | | | | | |
|---|---|---|---|---|---|---|
| Magnetic Effect | V_{Hx}¹ | V_{Hx}² | V_{Hy}¹ | V_{Hy}² | V_{Hz}¹ | V_{Hz}² |
| B_{z} ↑ | N/A | ↑ | ↑ | N/A | N/A | N/A |
| B_{y} ↑ | ↑ | N/A | N/A | N/A | ↑ | N/A |
| Bₓ ↑ | N/A | N/A | N/A | ↑ | N/A | ↑ |

In view of FIG. 5 and Table 1, V_{Hx}¹ may correspond to first X-transducer 322A, V_{Hx}² may correspond to second X-transducer 328A, V_{Hy}¹ may correspond to first y-transducer 316A, V_{Hy}² may correspond to second y-transducer 322B, V_{Hz}¹ may correspond to first z-transducer 316B, and V_{Hz}² may correspond to second z-transducer 328B. The relative behaviors of the magnetic fields as they move transverse across a patient chest cavity, up the chest cavity, or down the chest cavity and the relationship to the functional algorithm are defined further below.

Referring now to FIGS. 6A-C, the behavior of the magnetic fields may be described by a series of points along a movement of the locatable tip 250 of the catheter 200 (FIG. 1). The series of points may represent the movement in two dimensions. In some embodiments the catheter is a peripherally inserted central catheter (PICC) placed over a guidewire. Movement of the locatable tip 250 of the catheter 200 may be approximated as either moving across (FIG. 6A), up (FIG. 6B), or down (FIG. 6C) the patient 101 (FIG. 1) relative to a previous position of the locatable tip 250. Each of these movements may result in different behaviors from the three sensor clusters 310A-C (FIG. 3). In some embodiments the sensor clusters may be placed at the SVC or at the Atrioventricular (AV) node of the patient 101.

In each case relative points have been labeled P1, P2, P3, and P4 on the graphs, indicating points of interest. (P1) Represents the distant approach to the device in all cases. (P2) Represents the point of nearest approach to all sensors but particularly the proximal sensor cluster the PICC path generally denoted SI. (P3) Represents a point just after the branching above the AV node and several centimeters after the entry into the direction chosen. (P4) Represents a point after the branching point above the AV node and represents the terminal point of analysis. In each case each of the three sensor clusters will experience a change in the magnetic field of the system as a function of those three points.

The algorithm shall enumerate the three sensor clusters and the individual sensors at continuous points along this path. Points chosen are here for representation and explanation only.

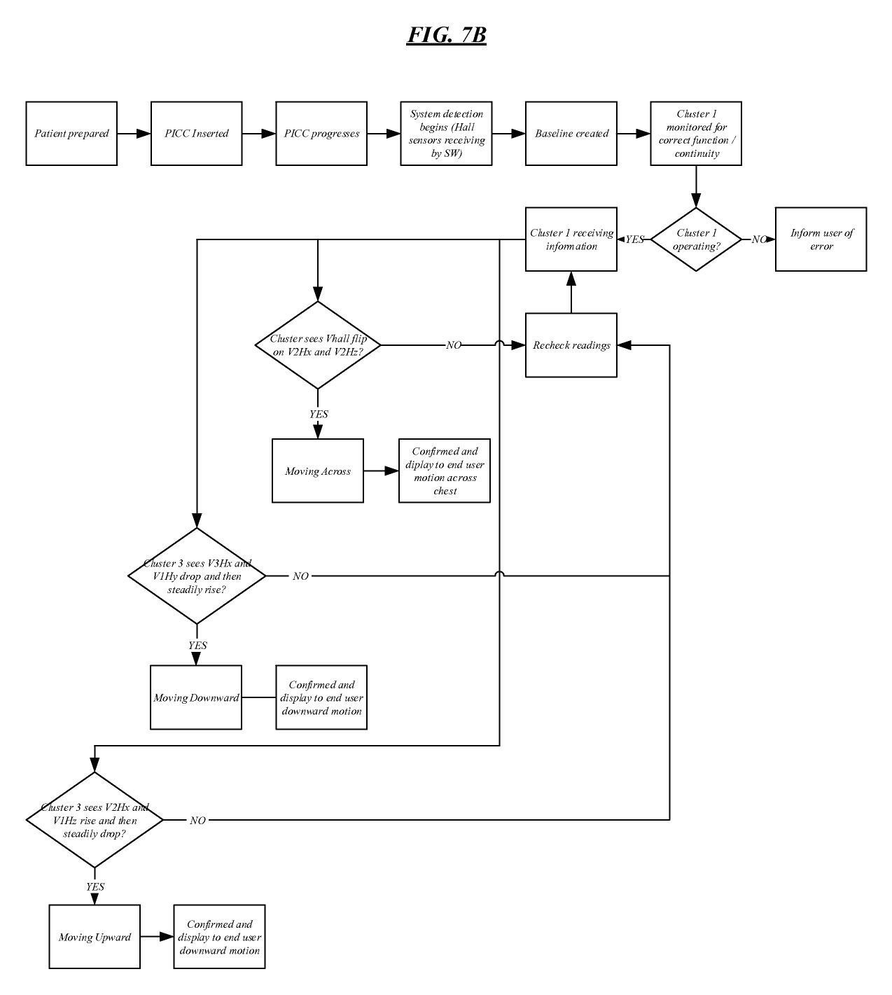

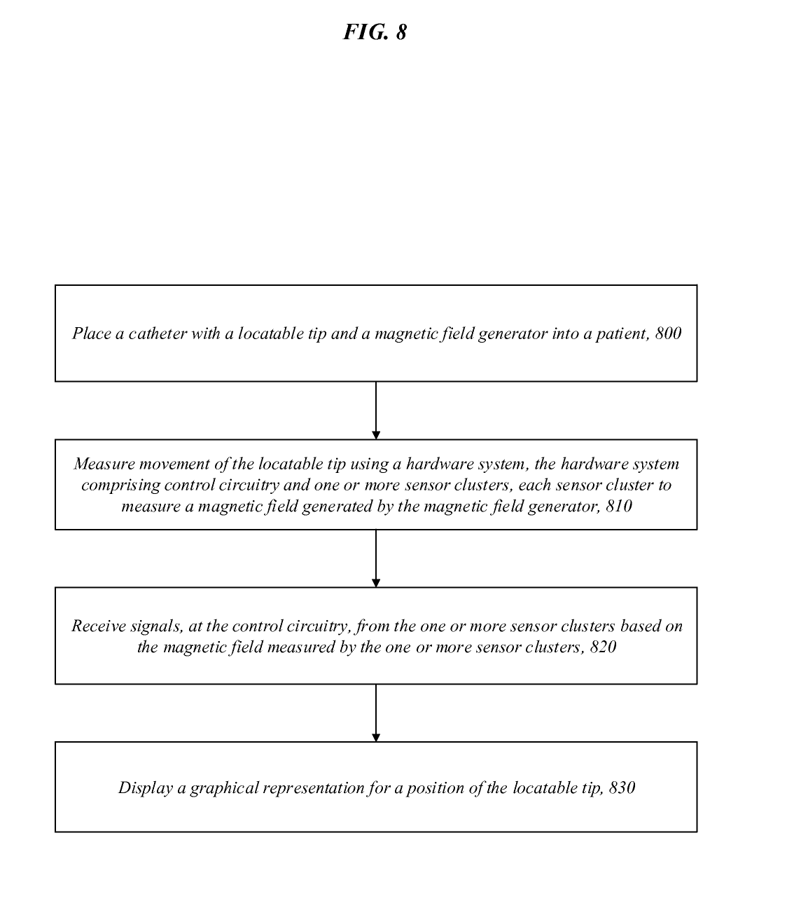

As the PICC or guidewire are advanced, the system has two means of analyzing the motion rise and fall in field strength and two associated algorithms for determining motion type described herein and illustrated by FIGS. 7A-B.

Motion away or toward the point by means of translation motion will be modified in terms of the field strength fall off per equation (10).

$$B α \frac{K}{{r}^{2}} . \qquad ( 10 )$$

This is a consequence of the magnetic field strength distant from a point by a linear distance, r. If on the other hand the field is modified not by the change in distance but is instead modified by the change in orientation, the individual components of the field will be modified in the rotation of the vector components by the sin or cos of the angle between the initial parallelism and the new orientation as seen in equation (11).

B→B₀ sin θ (11)

The algorithm has built in the following progress matrix:

B field progress path for Up the patient:

| Point in path/B | | | |
|---|---|---|---|
| field effects | Sensor Cluster 1 B effect | Sensor Cluster 2 B effect | Sensor Cluster 3 B effect |
| 1 | >Nominal (No change) | >Nominal (No change) | >Nominal (No change) |
| 2 | $> Increase by \frac{K}{{r}^{2}} ratio of {B}_{y}$ | $> Increase by \frac{K}{{r}^{2}} ratio of {B}_{y}$ | $> Increase by \frac{K}{{r}^{2}} ratio of {B}_{y}$ >Decrease by B₀ sin θ function of B_{z} |
| 3 | $> Decrease by \frac{K}{{r}^{2}} ratio of {B}_{y}$ | $> Increase by \frac{K}{{r}^{2}} ratio of {B}_{y}$ | $> Decrease by \frac{K}{{r}^{2}} ratio of {B}_{y}$ |
| | >Decrease by B₀ sin θ function | >Decrease by B₀ sin θ function | >Decrease by B₀ sin θ function |
| | of B_{y} | of B_{y} | of B_{y} |
| | >Increase by B₀ sin θ function of B_{z} | >Increase by B₀ sin θ function of B_{z} | $> Decrease by \frac{K}{{r}^{2}} ratio of {B}_{z}$ |
| | | | Increase by B₀ sin θ function |
| | | | of B_{z} |
| 4 | $> Decrease by \frac{K}{{r}^{2}} ratio of {B}_{y}$ | $> Decrease by \frac{K}{{r}^{2}} ratio of {B}_{y}$ | $> Decrease by \frac{K}{{r}^{2}} ratio of {B}_{y}$ |
| | >Decrease by B₀ sin θ function | >Decrease by B₀ sin θ function | >Decrease by B₀ sin θ function |
| | of B_{y} (to near zero) | of B_{y} (to near zero) | of B_{y} (to near zero) |
| | >Increase by B₀ sin θ function of B_{z} | >Increase by B₀ sin θ function of B_{z} | $> Decrease by \frac{K}{{r}^{2}} ratio of {B}_{z}$ |
| | $> Decrease by \frac{K}{{r}^{2}} ratio of {B}_{z}$ | | >Increase by B₀ sin θ function of B_{z} |

B field progress path for across the patient:

| Point in path/B | | | |
|---|---|---|---|
| field effects | Sensor Cluster 1 B effect | Sensor Cluster 2 B effect | Sensor Cluster 3 B effect |
| 1 | >Nominal (No change) | >Nominal (No change) | >Nominal (No change) |
| 2 | $> Increase by \frac{K}{{r}^{2}} ratio of {B}_{y}$ | $> Increase by \frac{K}{{r}^{2}} ratio of {B}_{y}$ | $> Increase by \frac{K}{{r}^{2}} ratio of {B}_{y}$ >Decrease by B₀ sin θ function of B_{z} |
| 3 | $> Decrease by \frac{K}{{r}^{2}} ratio of {B}_{y}$ >Decrease by B₀ sin θ function of B_{y} >Increase by B₀ sin θ function of B_{z} | $> Increase by \frac{K}{{r}^{2}} ratio of {B}_{y}$ >Increase by B₀ sin θ function of B_{y} | Change in sign of B_{y} with equal value |
| 4 | $> Decrease by \frac{K}{{r}^{2}} ratio of {B}_{y}$ | $> Decrease by \frac{K}{{r}^{2}} ratio of {B}_{z}$ | $> Decrease by \frac{K}{{r}^{2}} ratio of {B}_{y}$ >Decrease by B₀ sin θ function of B_{y} $> Decrease by \frac{K}{{r}^{2}} ratio of {B}_{z}$ >Increase by B₀ sin θ function of B_{z} |

B field progress path for down the patient:

| Point in path/B | | | |
|---|---|---|---|
| field effects | Sensor Cluster 1 B effect | Sensor Cluster 2 B effect | Sensor Cluster 3 B effect |
| 1 | >Nominal (No change) | >Nominal (No change) | >Nominal (No change) |
| 2 | $> Increase by \frac{K}{{r}^{2}} ratio of {B}_{y}$ | $> Increase by \frac{K}{{r}^{2}} ratio of {B}_{y}$ | $> Increase by \frac{K}{{r}^{2}} ratio of {B}_{y}$ >Decrease by B₀ sin θ function of B_{z} |
| 3 | >Decrease by B₀ sin θ function of B_{y} >Increase by B₀ sin θ function of B_{z} | >Decrease by B₀ sin θ function of B_{y} >Increase by B₀ sin θ function of B_{z} | >Decrease by B₀ sin θ function of B_{y} $> Increase by \frac{K}{{r}^{2}} ratio of {B}_{y}$ >Increase by B₀ sin θ function of B_{z} $> Increase by \frac{K}{{r}^{2}} ratio of {B}_{z}$ |
| 4 | >Decrease by B₀ sin θ function of B_{y} (to near zero) $> Decrease by \frac{K}{{r}^{2}} ratio of {B}_{y}$ >Increase by B₀ sin θ function of B_{z} $> Decrease by \frac{K}{{r}^{2}} ratio of {B}_{z}$ | >Decrease by B₀ sin θ function of B_{y} (to near zero) $> Decrease by \frac{K}{{r}^{2}} ratio of {B}_{y}$ >Increase by B₀ sin θ function of B_{z} | >Decrease by B₀ sin θ function of B_{y} (to near zero) >Increase by B₀ sin θ function of B_{z} $> Increase by \frac{K}{{r}^{2}} ratio of {B}_{z}$ |

The software will analyze this progress in real time, by means of the hall effects. Using the Table 1 above, each step in the change in the B field equates to a known change in the hall voltages. The above table may be reduced to the key elements of change that occur at points of transition between 2 and 3 and 3 and 4. Note there are never any changes in the direction sensing that may change the field in the x-direction (into and out of the chest cavity). This reduces the table in 2 to the table for the matrix in 2 seen below:

TABLE 2

| | Sensor Effect | | | | | | | | |
|---|---|---|---|---|---|---|---|---|---|
| Magnetic Effect | V_{Hx}¹ | V_{Hx}² | V_{Hx}³ | V_{Hy}¹ | V_{Hy}² | V_{Hy}³ | V_{Hz}¹ | V_{Hz}² | V_{Hz}³ |
| B_{z} ↑ | N/A | N/A | ↑ | ↑ | N/A | N/A | N/A | N/A | N/A |
| B_{y} ↑ | N/A | ↑ | N/A | N/A | N/A | N/A | ↑ | N/A | N/A |

Combine Table 2 with the effect of the progress path above for each of the directions to obtain the algorithm and compare only changing paths:

Algorithm behavior for Paths Cluster 1: (Cluster 1 carries no information alone)

| | Sensor Effect | | | | | | | | |
|---|---|---|---|---|---|---|---|---|---|
| Magnetic Effect | V_{Hx}¹ | V_{Hx}² | V_{Hx}³ | V_{Hy}¹ | V_{Hy}² | V_{Hy}³ | V_{Hz}¹ | V_{Hz}² | V_{Hz}³ |
| Upward path | ---/---/--- | ↑↓↓ | _↑_{—} | _↑_{—} | ---/---/--- | ---/---/--- | ↑↓↓ | ---/---/--- | ---/---/--- |
| Across path | ---/---/--- | ↑↓↓ | _↑_{—} | _↑_{—} | ---/---/--- | ---/---/--- | ↑↓↓ | ---/---/--- | ---/---/--- |
| Downward path | ---/---/--- | ↑↓↓ | _↑_{—} | _↑_{—} | ---/---/--- | ---/---/--- | ↑↓↓ | ---/---/--- | ---/---/--- |

Algorithm behavior for Paths Cluster 2: (Cluster 2 carries)

| | Sensor Effect | | | | | | | | |
|---|---|---|---|---|---|---|---|---|---|
| Magnetic Effect | V_{Hx}¹ | V_{Hx}² | V_{Hx}³ | V_{Hy}¹ | V_{Hy}² | V_{Hy}³ | V_{Hz}¹ | V_{Hz}² | V_{Hz}³ |
| Upward path | ---/---/--- | ↑_↓ | _↑↑ | _↑↑ | ---/---/--- | ---/---/--- | ↑_↓ | ---/---/--- | ---/---/--- |
| Across path | ---/---/--- | ↑↑_{—} | _{——}↓ | _{——}↓ | ---/---/--- | ---/---/--- | ↑↑_{—} | ---/---/--- | ---/---/--- |
| Downward path | ---/---/--- | ↑↓↓ | _↑↑ | _↑↑ | ---/---/--- | ---/---/--- | ↑↓↓ | ---/---/--- | ---/---/--- |

Algorithm behavior for Paths Cluster 3: (Cluster 3 uniquely identifies across, up, and down)

| | Sensor Effect | | | | | | | | |
|---|---|---|---|---|---|---|---|---|---|
| Magnetic Effect | V_{Hx}¹ | V_{Hx}² | V_{Hx}³ | V_{Hy}¹ | V_{Hy}² | V_{Hy}³ | V_{Hz}¹ | V_{Hz}² | V_{Hz}³ |
| Upward path | ---/---/--- | ↑↓↓ | ↓_{——} | ↓_{——} | ---/---/--- | ---/---/--- | ↑↓↓ | ---/---/--- | ---/---/--- |
| Across path | ---/---/--- | ↑Flip↓ | ↓_{——} | ↓_{——} | ---/---/--- | ---/---/--- | ↑Flip↓ | ---/---/--- | ---/---/--- |
| Downward path | ---/---/--- | ↑_↓ | ↓↑↑ | ↓↑↑ | ---/---/--- | ---/---/--- | ↑_↓ | ---/---/--- | ---/---/--- |

An algorithm based on the information above is illustrated in FIG. 7A. Based on this algorithm, the unique rise and fall of the voltage sensors within the clusters can be used to determine the direction of motion of the PICC in the body. In some embodiments the key first algorithm elements may include one or more of the following: monitoring the rise and fall of relative voltage cluster within the Hall sensor to determine the direction of motion; monitoring the sign (direction) of the Hall voltage and using their relative direction to determine the direction of motion in the body; and monitoring the clusters as units for redundancy in measurements.

In some embodiments the algorithm may have a feedback loop to the magnetic field control port 350 (FIG. 3) to modify the magnitude and or direction of the magnetic field generated by electromagnet 261B (FIG. 2B). In an alternative embodiment the algorithm may not use redundancy in the sensor clusters. In this embodiment the system need only have a single sensor placed in the location of sensor cluster 2 or 3, (below and to the left side of the AV node). In this mode the system can follow a simpler algorithm as illustrated in FIG. 7B. In this alternate single sensor cluster algorithm there is no redundancy check with new clusters. Otherwise all algorithm behaviors derived for sensor cluster 3, now applied to single sensor cluster 1 in location of cluster 3 will operate independently.

In some embodiments an additional algorithm function may be added to the algorithms described above for single and multiple sensor functions. The above tables in the exemplary embodiments indicate not only the rise and fall of the associated Hall voltages but also the associated type of rise and fall with the use of Eqns. (10 & 11). This allows the CPU to perform Chi Squared fit on the real time data to determine if the rise and fall of the type of equation (10) or (11). The real time data may include sets of data output by one or more sensors. Through the use of this fit, the system would be able to determine the direction of motion by directionally monitoring the translation versus the rotation of the PICC as it takes place in real time.

The key elements to the additional algorithm function described above may include one or more of the following: monitoring the chi squared fit function to determine direction of motion of the placed PICC; monitoring for K/r² fit and function of the system to determine rotational motion; use of these function fits to uniquely determine motion of the PICC; and use of these function fits to double check or corroborate otherwise determined motion of the PICC.

Referring now to FIG. 8, a logic diagram illustrating an exemplary method of calibrating a cutting device will be described in greater detail. As shown in block 800, a catheter with a locatable tip and a magnetic field generator can be placed into a patient. In various embodiments, the catheter is a peripherally inserted central catheter (PICC). In some embodiments, the magnetic field generator may include a permanent magnet. In various embodiments, the magnetic field generator may include an electromagnet. In block 810, a hardware system can be used to measure movement of the locatable tip. The hardware system can include one or more sensor clusters and control circuitry. Each sensor cluster may measure a magnetic field generated by the magnetic field generator. In various embodiments, the one or more sensor clusters may include three directional sensors to measure the magnetic field. In some embodiments the control circuitry may alter the magnetic field generated by the magnetic field generator. At block 820, the control circuitry can receive signals from the one or more sensor clusters based on the magnetic field measured by the one or more sensor clusters. A graphical representation for a position of the locatable tip may then be displayed at block 830. In some embodiments the position of the locatable tip may be displayed on a user interface.

Some embodiments of the disclosed device may be implemented, for example, using a storage medium, a computer-readable medium or an article of manufacture which may store an instruction or a set of instructions that, if executed by a machine (i.e., processor or microcontroller), may cause the machine to perform a method and/or operations in accordance with embodiments of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

References to “one embodiment,” “an embodiment,” “example embodiment,” “various embodiments,” etc., indicate that the embodiment(s) of the invention so described may include a particular feature, structure, component, or characteristic, but not every embodiment necessarily includes the particular feature, structure, component, or characteristic. Further, repeated use of the phrase “in one embodiment” or “in some embodiments” does not necessarily refer to the same embodiment(s), although it may.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

1. A catheter location device, comprising:
one or more sensor clusters, each sensor cluster to measure a first set of voltage values when a locatable tip of a catheter is at a first position and a second set of voltage values when the locatable tip in at a second position; and
control circuitry to include a processor communicatively coupled to the one or more sensor clusters and a non-transitory computer-readable medium, the processor executing instructions stored on the non-transitory computer-readable medium to:
calculate the second position based in part on the first position and in part on a change between the first set of voltage values and the second set of voltages, and
store the second position of the locatable tip to the non-transitory computer-readable medium.

2. The device of claim 1, wherein each sensor cluster includes at least two directional sensors.

3. The device of claim 2, the directional sensors each comprising a current block and first and second transducers.

4. The device of claim 3, the first transducer to measure a first voltage difference across a first dimension of the conductor block and the second transducer to measure a second voltage difference across a second dimension of the conductor block.

5. The device of claim 4, wherein the first and second dimensions of the conductor block are generally orthogonal with respect to each other.

6. The device of claim 4, the first set of voltage values to include the first and second voltage differences when the locatable tip is at the first position and the second set of voltage values to include the first and second voltage differences when the locatable tip is at the second position.

7. The device of claim 1, the processor executing instructions stored on the non-transitory computer-readable medium to communicate the second position relative to the first position via a user interface.

8. The device of claim 7, comprising a housing to enclose one or more of the plurality of sensor clusters, the processor, the non-transitory computer-readable medium, and the user interface.

9. The device of claim 5, wherein the plurality of sensor clusters are located above a chest cavity of a patient when the second position is calculated.

10. A catheter location system, comprising:
a catheter with a locatable tip, the locatable tip including a magnetic field generator; and
a hardware platform, the hardware platform to include:
one or more sensor clusters, each of the sensor clusters to measure a magnetic field generated by the magnetic field generator, and
control circuitry to receive signals from the one or more sensor clusters based on the magnetic fields measured by the first, second, and third sensor clusters and display a graphical representation for a position of the locatable tip.

11. The catheter location system of claim 10, wherein the hardware platform is conductively coupled to the magnetic field generator, the magnetic field generator to include an electromagnet.

12. The catheter location system of claim 11, the control circuitry to control a magnetic field generated by the magnetic field generator via a control link.

13. The catheter location system of claim 12, wherein the electromagnet receives power via the control link.

14. A method of locating a catheter comprising:
placing a catheter into a patient, the catheter having a locatable tip and a magnetic field generator;
measuring movement of the locatable tip using a hardware system, the hardware system comprising control circuitry and one or more sensor clusters, each sensor cluster to measure a magnetic field generated by the magnetic field generator;
receiving signals, at the control circuitry, from the one or more sensor clusters based on the magnetic field measured by the one or more sensor clusters; and
displaying a graphical representation for a position of the locatable tip.

15. The method of claim 14, wherein the graphical representation for the position of the locatable tip is displayed on a user interface.

16. The method of claim 14, wherein the control circuitry alters the magnetic field generated by the magnetic field generator.

17. The method of claim 14, wherein the catheter is a peripherally inserted central catheter (PICC).

18. The method of claim 14, wherein the magnetic field generator includes a permanent magnet.

19. The method of claim 14, wherein the magnetic field generator includes an electromagnet.

20. The method of claim 14, wherein each of the one or more sensor clusters includes three directional sensors to measure the magnetic field.
